(19) United States
(12) Patent Application Publication
(10) Pub. No.: US 2010/0190129 A1
(43) Pub. Date: Jul. 29, 2010

(54) COMBINATION DENTAL HAND TOOL

(76) Inventor: Mony Paz, Auckland (NZ)

Correspondence Address: YOUNG & THOMPSON, 209 Madison Street, Suite 500, Alexandria, VA, 22314, US

(21) Appl. No.: 12/376,703

(22) PCT Filed: Aug. 6, 2007
(86) PCT No.: PCT/NZ07/00207

§ 371 Date: Feb. 6, 2009

(30) Foreign Application Priority Data
Aug. 8, 2006 (NZ) 549027
Mar. 29, 2007 (NZ) 554257

(51) Int. Cl. A61B 1/24

(52) U.S. Cl. 433/29

(57) ABSTRACT

A hand-held self-powered combination dental tool is capable of performing multiple concurrent functions including suction, retraction, and intra-oral lighting so that dental treatment can be carried out easily under good lighting. (A suction connection is required). Another lighting mode (blue to ultraviolet) also allows inspection of the oral cavity for lesions such as hyperplasia or neoplasia using local changes in tissue fluorescence as an indicator. Internal batteries are rechargeable. Measurements of tissue fluorescence can be made and reported using internal detection and digital measurement.

### FIELD OF THE INVENTION

The present invention relates to a dental tool; in particular to a hand-held dental tool having concurrent support functions for use during a treatment procedure; the functions including suction, retraction, and intra-oral lighting.

1. Background of the Invention

When performing dental work on a patient, the dentist and dental assistant use a range of tools. Necessary tools include drills, mirrors, scalers, probes, excavators, other dental implements and suction tools. Since the patient's lips comprise a limited aperture (the “oral aperture”), it is important to minimise the number and size of the tools in use at any one time to the absolute minimum.

Suction tools are very frequently used by the dentist or dental assistant to remove water, debris, and other matter from the oral cavity during surgery in order to maintain a relatively clean environment. Further, there has to be enough space remaining through the oral aperture in order to see the site of the dental work and to let external light into the mouth. The overhead light is normally a filament lamp having a dichroic filter in the reflector, to prevent the oral aperture from being heated. It is difficult to direct all the light into the oral cavity without shadows or high contrast between the lips and the interior. Some working positions adopted by a dentist or assistant will inevitably block the external light.

It is desirable to use a retractor, often made of a rigid material, to hold the oral tissues away from the work site, and to restrain the patient's tongue which is very mobile and is likely to be damaged—cut or abraded—by dental tools.

Apart from performing dental work, the dentist (or a hygienist, acting as a screening person), often looks for signs or early signs of abnormalities in the lining of the oral cavity. Early detection of cancer is always preferable and is currently encouraged by public health bodies and medical insurance companies. Diseased tissue is relatively difficult to identify under plain white light without filters, and requires significant experience. Because of reasons that are not fully understood, normal and diseased tissues have different optical properties, particularly but not solely manifested as variations in colour or intensity of autofluorescence. It is known that many if not all lesions such as hyperplasia or neoplasia will exhibit changes in tissue autofluorescence, or may take up supravital dyes to an extent different from normal epithelia, even if their appearance under white light is unremarkable. More specifically, tissue suspected of being dysplastic or tending to become an invasive carcinoma is associated with progressively reduced auto-fluorescence.

One useful way to screen patients is by using light having a selected wavelength or range of wavelengths capable of exciting or inducing fluorescence in tissue or in added dyes (such as green, blue or ultraviolet light), and to view the epithelium through optical filters of other selected wavelengths or ranges (such as yellow, orange or red filters) that effectively block the inducing light but transmit the resulting fluorescent light. These are sometimes called “low-pass” optical filters.

2. Prior Art

Some aspects of the combination tool of the present invention are known from the patent literature. “Aspect” refers to an aspect of the present invention.

Aspect 1: a transparent, hollow suction tip for lighting. A replaceable transparent suction tip is found in U.S. Pat. No. 4,872,837 Issalene et al, and in WO99/47068 Davis et al. Both include means to aim light beams along the suction tube which acts as a light pipe. Issalene et al allows the suction tip to rotate within the housing, while Davis et al direct most of their claims to means to fix it in place. Korff DE 3939859 uses an external fibre optic guide running along the outside of a suction tip. That, unlike the previous two, is incompatible with disposability and easy, cheap replacement with a sterile suction tip. See also U.S. Pat. No. 5,931,670 Davis (re-used suction tip plus fibre optic light source). Provision of white light emerging at the “business end” of other dental tools such as drills is covered in a number of publications such as GB1280339 Everett (1972) for various fibre optics-lit tools.

Aspect 2: a transparent retractor on the end of the suction tip. No citations have been found for water-clear or transparent but coloured retractors. US2004/0254478 (de Josselin de Jong) describes a dedicated light-pipe for dental use which may carry UV light and which may have small filters attached but the primary purpose is to illuminate teeth and no retractor-like function is described.

Aspect 3. No citations have been found for shining blue to ultra-violet light by suction tip means into the mouth. The “Kniseley” quartz light-pipe was developed for experimental micro-surgery about 60 years ago. It is a tapering quartz rod having a central duct, and is capable of conducting light and cooling liquid to a working site and, being made of quartz, would be capable of transmitting ultra violet light.

Aspect 4. Shining ultra-violet light by non-suction tip means into the mouth. JP 5049681 provides a sterilising tube which is placed in the oral cavity and emits a sterilising type of ultraviolet light, the option of shining ultra-violet light along the suction tip into the mouth. WO94/09718 Kurze describes a floor-mounted light source including an ultraviolet source and holder for a moveable arm that terminates with a rubber-elastic (non-transparent) suction tip. Davis et al do not teach ultra-violet light. US2004/0254478 (de Josselin de Jong) describes a dedicated light-pipe for dental use which may carry UV light.

Aspect 5. Shining ultra-violet light into the mouth for the purpose of visual screening for certain types of cancer. U.S. Pat. No. 6,325,623 Melynk et al take a conventional light-curing lamp for dentistry (which emits a strong green-blue light, and mount a low-pass filter (for filtering out exciting light and leaving only light emitted by fluorescence) around the tip; not very much different to the circular orange guards, used for the assistant's protection against bright light that are conventionally provided with this equipment. WO2005/099563 LED MEDICAL describes an externally mounted shroud for shielding the mouth from ambient light, for examination of the skin and lips as well as the oral cavity. US2004/0254478 (de Josselin de Jong) describes a dedicated light-pipe (which is single-purpose; not also a suction tip, drill, or any other device. It includes a surrounding low-pass filter. WO2003/071953 Wilder-Smith et al relates to supra-vital staining with a fluorescent dye, then use of an excitation light of about 405 nm in order to excite fluorescence visualised at or about 635 nm. Melynk et al referred to use of various supra-vital dyes including the 5-amino-levulinic acid of Wilder-Smith et al.

3. Object

It is therefore desirable to provide a more convenient way to carry out most or all of the dental functions listed above under “Background” using a single tool, while preserving the necessary attributes such as sterility, for reasons of simplicity, speed and economy of time; or at least to offer the relevant public a useful choice.

### SUMMARY OF THE INVENTION

In a first broad aspect, the invention provides a combination dental hand tool for several separate or often concurrent purposes by a user during a dental treatment procedure carried out on a patient, characterised in that the tool has a self-contained hand-held body containing an energy source and illumination means and having a first coupling means at a rear end for coupling to an existing flexible vacuum line and a second coupling means at a front end for firmly yet removably holding a transparent and water-clear suction tip capable of bearing a retractor; the retractor comprising a small transparent rigid flap projecting to a selectable side of the tip; the tip and the retractor together capable when in use of being positioned by the user for a first purpose of pushing and holding soft parts of the oral cavity away from a treatment area; the tool also having the second purpose of suction of materials from the patient's mouth through a duct passing along the length of the suction tip and into the vacuum line; the tool also having the third purpose of controllable illumination of the patient's mouth with white light that is transmitted from an illumination source through the suction tip which serves as a light-pipe to carry light into the vicinity of the treatment area.

Optionally the tool and the suction tip include co-operative light entrainment or concentrating means that collect light emitted within the tool and direct said light along the suction tip towards the distal end.

Preferably, the body is reusable.

In a first related aspect, the illumination means comprises at least one solid-state emitter of coherent or incoherent light working within a specified range of wavelengths, and provided with a working current from the energy source within the tool.

Preferably the specified range includes blue to ultra-violet light of between about 480 and about 360 nm so that fluorescence is induced in or on tissues in the vicinity of the treatment area and so that the resulting fluorescence facilitates recognition of abnormal tissues within the patient's mouth.

In a second related aspect, the transparent retractor is dyed with a selected dye in order to selectively filter out (block) the blue to ultra-violet light yet transmit those visible wavelengths emitted by induced fluorescence of tissue, so that the presence or absence of tissue autofluorescence can be evaluated by using the retractor to block the fluorescence-inducing light and seeing any differences of fluorescence through the retractor.

Alternatively, the emitted and filtered wavelength ranges are selected in order to induce and reveal fluorescent light emitted by selected supravital stains rather than by autofluorescence, so that the procedure of recognition of abnormal tissues is enhanced.

Preferably the retractor is capable of being taken off the suction tip, rotated around the suction tip and replaced in a different attitude.

In a third related aspect, a new suction tip and retractor is supplied in a clean pack for each use, thereby reducing a risk of transmission of disease from patient to patient.

Preferably the illumination source is cooled during use by drawing a flow of air over at least one surface in thermal contact with the illumination means and into the vacuum line, thereby also maintaining movement of liquids along the vacuum line.

In a fourth related aspect, the tool further includes a fourth purpose of detecting and measuring that visible light caused by induced fluorescence after transmission back from the distal end of the suction tip to light measuring means in the tool and then to processing means capable of measuring and reporting abnormal amounts of fluorescence even in the absence of direct inspection of suspected tissues.

Optionally, a suction tip used in this mode lacks a central duct and is a solid light-pipe.

Optionally, the blue-ultraviolet source is repeatedly turned on then off in a cyclic manner and the light measuring means is provided with processing means controlled so as to subtract a first reading taken when the source capable of inducing fluorescence is off from a second reading obtained when the source is on, hence rendering measurements more independent of ambient light.

As a further option, returned light from at least one source of light having a wavelength in a range lying within a broad range of from about 500 to about 1000 manometers wavelength—is generated in a cyclic manner, passed down the suction tip and the reflected amount of light is measured and used by the processing means in order to enhance differentiation of normal from abnormal tissues. For example, blue/UV (as previously described in this section), orange, and infra-red colours may be generated in turn and the returned light measured and set of measurements stored.

Preferably the processing means comprises a micro-computer within the hand tool and the readings are stored and provided in digital form.

Alternatively, processing may be done with analogue electronics.

Preferably also, the processing means includes memory means capable of passing stored tissue fluorescence results to a computer through a compatible wired connection; the wired connection also being capable of recharging the energy source within the hand tool.

### Preferred Embodiments

The description of the invention to be provided herein is given purely by way of example and is not to be taken in any way as limiting the scope or extent of the invention. Throughout this specification, unless the text requires otherwise, the word “comprise” and variations such as “comprising” or “comprises” will be understood to imply the inclusion of a stated integer or step or group of integers or steps but not the exclusion of any other integer or step or group of integers or steps.

### BRIEF DESCRIPTION OF THE DRAWINGS

Preferred embodiments of the invention will be described in relation to the accompanying drawings, of which:

FIG. 1a: is an external view of the dental tool with attached suction tip and retractor.

FIG. 1b: is a sectional, view of an early version of the dental tool.

FIG. 2: shows a retractor in face view, showing further details.

FIG. 3: an example suction tip, in side view.

FIG. 4: shows sections A-A through example suction tips.

FIG. 5: shows a non-suction kind of “suction tip”.

FIG. 6: shows use of mirrors and lenses used to direct bright LED light along a suction tip.

FIG. 7: is a block diagram of an electronic control and optional measurement circuit.

FIG. 8: shows an exploded view of a combination hand-held dental tool.

FIG. 8a: shows a variant of FIG. 8, using forward-facing LEDs.

In this text, the clear plastic light guide extending from the front of the combination dental hand tool is called a “suction tip” because most, but not all versions according to this invention include an internal duct that allows fluids and detritus to be withdrawn from the patient's mouth in the usual way. Some of these light guides lack a central duct (20a) but retain other functions and are still called “suction tips” in this specification.

### DETAILED DESCRIPTIONS

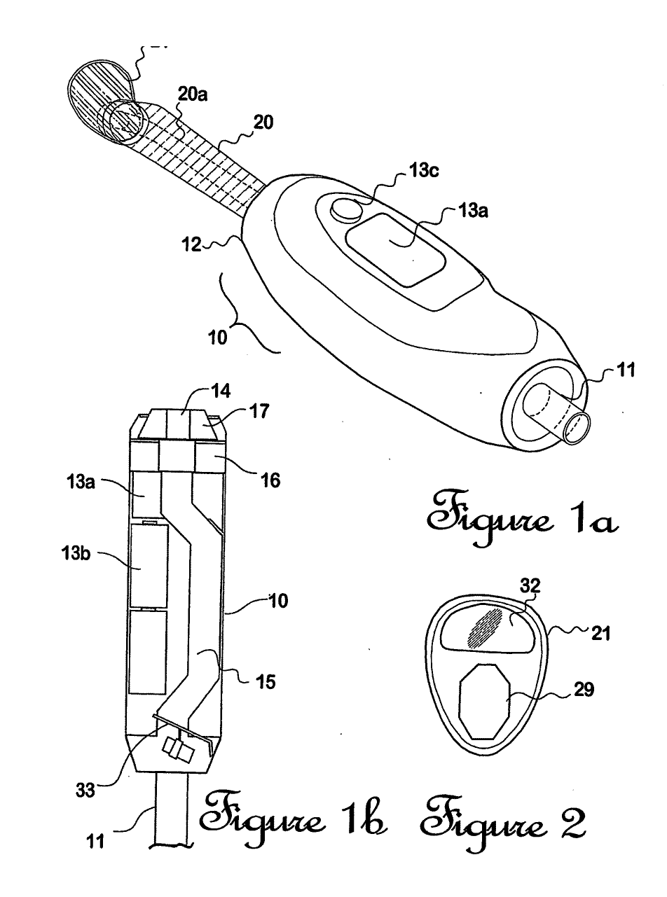

The invention is a multi-purpose hand-held dental tool or implement. FIGS. 1a (perspective) and 1b (section) show preferred embodiments, having a main body/housing 10. In FIG. 1a, a disposable and removable, centrally ducted suction tip 20 is shown in place and ready for use. Preferably the suction tip is also provided with a disposable and removable retractor 21 (for details see Aspect 2). The suction tip is made of a water-clear plastics material that is also transparent to ultraviolet light over a desired range, to be used for screening purposes (see below). At the front 14, the tool carries means 17 for temporarily yet firmly mounting a disposable suction tip 20 having a central duct 20a that is joined to the vacuum inlet 11 placed at the back of the hand-held tool by way of the internal tube 15, for withdrawing material from the patient's mouth.

The body 10 contains controllable lighting means in a mount 16, an energy source (such as lithium hydride rechargeable batteries 13b), the suction pipe 15 and optional returned-light analysis means 13a (circuit board in FIG. 8, comprised of electronics) and display means 13d.

The body presents a mode control means, such as a press-button switch 13c, and a window for viewing an optional display 13d. The tool is easily incorporated into the range of equipment used in a typical dental surgery by being connected at the standard fitting 11 at the rear to an existing suction line or vacuum line. The version shown in FIG. 1b shows a spring-loaded valve 33 which when opened by the user causes the suction tip 20 to be connected to the suction line. Such a valve is usually provided as part of the existing suction line, to be connected at the pipe 11 at the rear of the dental tool.

The main body 10 may be used repeatedly. The batteries 13b within may be recharged in situ, using the computer connector (USB or similar; 13U in FIG. 8) or by connecting an external source, and/or fresh batteries may be installed. Disposable suction tips 20 (that are provided in more than one shape) and compatible retractors 21, 21A are supplied for use with the main body 10. A new set is highly preferred for each patient, principally to avoid transferring disease including without limitation the HIV virus from one patient to another and also so that fresh, clean, unblemished optical-grade surfaces are provided.

A replaceable transparent suction tip (20—FIG. 1, FIG. 8) according to the invention is typically about 70-100 mm long and has walls that are relatively thick about the central duct 20a. It is made of a visible and UV-transparent and water-clear material. Preferably the material is a strong plastic that can be injection-moulded. Preferably the material is bio-degradable so long as it retains strength until after use. Many commercial plastics deliberately include added ultra-violet absorbing materials because they are intended for either protection from the outside world or are intended to weather well when exposed to the sun. It is useful for the present invention to avoid these. Samples should be tested in case of inadvertent UV absorbtion properties. Appropriate suction tip plastic materials include acrylic; suitable if the wavelength is longer than 320 nm, polycarbonate (suitable if the wavelength is longer than 400 nm), or another injection mouldable or castable water-clear, transparent, non-brittle plastics material or alloy of plastics. A refractive index greater than that of water is desirable.

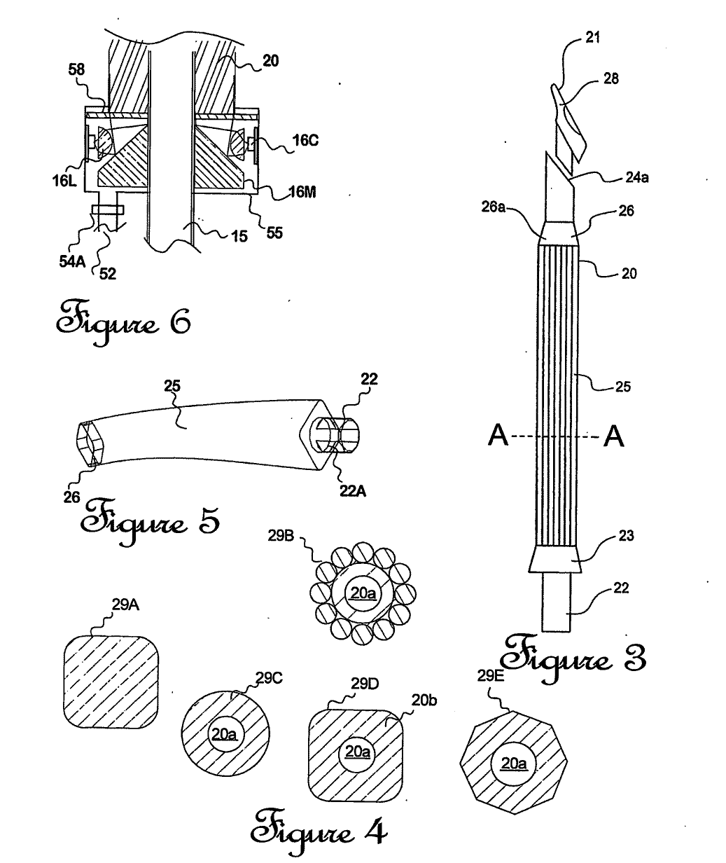

As previously described, UV or blue light or a mixture is emitted by selected LEDs and the light is directed or reflected, using total internal reflection along the suction tip so that it emerges at or near the end. Near the tip, there is mounting means for a retractor that is preferably indexed so as to prevent the retractor from spinning around on the tip if torque is applied. FIG. 4 shows some versions of a cross-section made at about section A-A of FIG. 3. Versions 29C, 29D and 29E are basic. These are rods having a round or polygonal outline, a relatively thick wall 20b, and a central duct 20a. (The central duct 20a may be gradually tapered; expanded at each end, for the purpose of being releasable from the mould). Use of a hexagonal or octagonal outline or the like for the distal end of the suction tip lets the user adjust the relative orientation of a matching retractor into 6 or 8 different attitudes, which may be fitted over a slightly tapered distal end and become locked in place. Version 29A, and FIG. 5, as shown, are of a solid, rounded-edge square-section light pipe.

This version illuminates and returns reflected light better but of course cannot suck. Version 29B represents the particular suction tip shown in side view in FIG. 3. This comprises a trial version having optically non-contiguous light pipes around the periphery of the central core; all made of transparent and water-clear plastic by injection moulding. The prototype of this version of the suction tip 20 in FIG. 3 used a bundle of end-polished light conducting rods 25 placed around the body of the suction tip and preferably bonded in place by a resin such as “Araldite” which retains the optical integrity (for total internal reflection) of the bonded body of plastic. This would allow the use of a strong duct wall. The light conducting rods 25 are optically coupled to the concentrator 23 and transfer light emitted therefrom along the length of the suction tip to an optically connected or adjacent section 26 so as to couple as much light as possible into the part 26 which is also frustoconical in shape and which channels the light from the light conductors 25 towards the inner tube of the open end of the suction tip. If diffusing material is included (which may be done just at or near the tip) it serves as an origin of scattered light. The escaping light illuminates the oral cavity surrounding the suction tip and some light escapes at the end of the tip 24a into the oral cavity where it is placed. An injection moulding procedure can place two kinds of clear plastics against each other in order to simulate this hand-built prototype.

A variety of suction tip lengths and shapes: straight, bent or curved are provided. See FIG. 8; left side at 20. The base of the suction tip preferably though not essentially includes keying means (FIG. 5; 22A for example) so that the entire tip does not spin about its insertion into the tool 10. A taper lock is a preferred attachment means; it is easy to clean, tolerant of moulding shrinkage, and would allow insertion at any orientation. The retractor 21 shown in FIG. 3 is attached by means of a sleeve that enters the end of the central duct 20a. Note the tapering collar or second concentrator 23 surrounding the protrusion 22 intended to fit within pipe 14, when the second concentrator 23 becomes mated with concentrator 17.

At this time, light-emitting diode (LED) technology appears to be the most appropriate type of lighting for use in the dental tool, for reasons including compactness, brightness, rapid switching, efficiency, availability of selected emitted colours including blue to ultra-violet, and at least partially collimated output being available. Organic LEDs have not been tested. Several versions of coupling of the light emitter to the base of the suction tip are provided in this Example.

The following description is of an example embodiment, although it must be realised that other arrangements of light emitters and light pipes may be used while remaining within the scope of the invention. The body of the dental tool includes one or more sources of light for the various colours, directed at and coupled to the base of the suction tip. Developments in relation to efficient light transfer down the suction tip are incomplete, so three variants are described here.

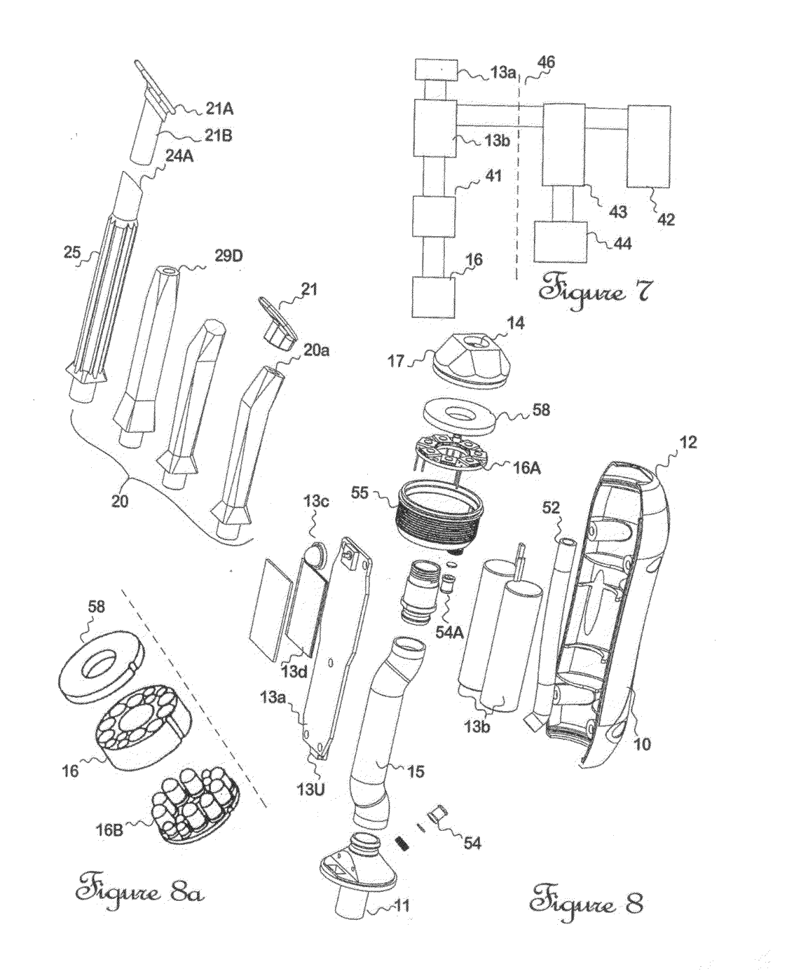

FIGS. 6, 8 and 8a show three ways to arrange LEDs to cause the emitted light to be directed down the suction tip. FIG. 8a shows one preferred variant of the ring block 16 which includes a plurality of apertures, such as 30a and 30b for holding lead-frame type LEDs 29. Preferably the apertures are as close to the axis as possible, and may be inclined slightly towards the axis rather than be drilled parallel to the axis. The ring block 16 is preferably made of a thermally conductive metal such as aluminum or copper and encircles the suction channel passing between pipe 15 and fitting 14 towards the front of the main housing 10, “High brightness” types of LED are preferred as far as current technology provides. Improvements in brightness are continually being made. The current example of the invention uses two UV LEDs that have their peak output at about 393-395 nm as well as two blue LEDs that have a peak at about 470 nm. Four larger 5 mm (T1 ¾) or 8 mm or 10 mm diameter apertures are provided for mounting larger LEDs for white light. Also, four 3 mm diameter apertures are provided 30b for mounting smaller 3 mm blue LEDs. Other numbers of holes, hole sizes and mounting arrangements are also covered by the preferred embodiment of this invention, as will be evident to those skilled in the art. For example Sensor Electronic Technology, Inc (South Carolina, USA) produces deep UV (in the range of 247-365 nm) LEDs in TOx transistor headers for better heat dissipation.

Mirrors may be used to bend the light, as shown in principle in FIG. 6 so that the light rays emitted radially inwards from each LED 16C are reflected forwards by a corresponding angled, polished metal mirror 16M after at least partial collimation by lens 16L (which may be included in the LED package) so that a substantial proportion of the rays pass along the light guide 20. The mirror itself may have curved faces that serve as partial or further collimators. The mirror might be formed within the end of component 20. The casing 55 is a polygonal box axially perforated to allow passage of the suction duct 15. The air-flow tube 52 (FIG. 7) is shown connected through a one-way valve 54A to the interior of the box, if air has been drawn in through apertures (not shown). The cooling air may instead be drawn over the outside of the box if it is thermally conductive. That would avoid the build-up of dust over optical parts. This diagram does not show details of coupling light into the suction tip, although it does include a window 58. The use of mirrors allows use of LEDs with large heat-sink skirts in conjunction with the central drain 15, and may avoid the use of frustoconical concentrators. “Luxeon™” family (Philips Lumileds) or similar LED assemblies which are made in 1, 3, or 5 watt types that rely on an included heat sink may be used in this version. Custom supply of an ultra-violet LED emitter in the “Luxeon™” package may also be desirable. The existing royal blue “Luxeon™” lamp has its peak of emitted light at around 455 nm; half-width 20 nm) which may suffice here.

Because single-chip white LEDs rely on a layer of fluorescent materials excited by blue to ultra-violet LED light to produce an orange light that merges with the blue to make white light, use of white LEDs in a fluorescence-measuring version as described below is probably not advisable although it may be possible. Instead, white light is preferably simulated with separate red, green and blue light emitters driven together. Selection of LEDs, and selected dyes used in the long-pass colour filter material used within the retractors allows the invention to be used with particular fluorescent dyes as well as with “natural” tissue autofluorescence. (The existing infra-red sensing option may be of use for some of those).

The option of surface-mount LEDs (as shown at 16A in the middle of FIG. 8) may be preferable because they are more effectively cooled through the copper lead that supports the emitting chip by soldering that lead to a circuit board having preferably relatively thick copper, routed so that the majority of the board surface (especially that surface connected to the lead) remains covered with copper. Through-hole connections to further copper surfaces underneath the board are also possible. The emitting crystals are closer to the external surfaces of the packages than for such as the ‘T1 ¾″’ packages. The copper surfaces may be in contact with an air-cooled, finned heat sink if required because it is likely that about one watt of heat will be generated. Again, cooling air that passes over the heat sink may be driven by the vacuum line. Forced cooling allows more power and/or a more compact body for the tool. The light sources 16 are powered and controlled by the power source 13a with electronics 13b and 41 (see FIG. 7).

A frustoconical concentrator 17 made of a water-clear and transparent refractile substance such as clear, UV-transparent plastic and with a central aperture 14 is provided in prototypes of the invention. The concentrator 17 is mounted so that it is abutted against a surface of the annular LED mount 16 and is aligned such that the central aperture is coaxial with the suction channel 15. Illumination made in the LEDs 16 is directed and channelled inwards due to the angular walls of the concentrator 17. The concentrator preferably protrudes at least some way pass the outer housing at the front end 12 of the main body 10. The frustoconical concentrator 17 may be coated on its exposed surfaces with an internally mirrored, opaque finish in order to minimise stray light escaping from this region. Optionally, a second conical concentrator 23 that forms part of the disposable suction tip is used in conjunction with the first concentrator 17. Preferably, the LED light sources are placed as close as possible to the central aperture so that the light is directed straight down the suction tip with minimal reliance on concentrators.

Referring to FIG. 3, the suction tip 20 comprises a connector 22 that is adapted to insert through the central aperture 14 of the concentrator 17 in the main body 10 in order to hold the suction attachment to the main body 10 and to couple the suction flow. “O”-ring or similar seals may be used to retain the parts. FIG. 5 shows one anti-rotation option—facets 22A, although a simple concentric taper, lacking preferred orientations, may be preferred. When the suction attachment is coupled to the main body 10 the connector 22 is sealed within the suction channel 15 that passes through the axis of the frustoconical concentrator 17. The corresponding concentrator 23 on the suction tip 20 abuts against the concentrator 17 so that light which is channelled through the concentrator 17 in this arrangement will continue into the concentrator 23 of the suction attachment and pass along the suction tip.

The suction attachment 20 comprises a central tubular member ending, at its distal end, in an opening 24 that is cut off at an angle 24a, useful when the tip of the attachment 20 is inserted into the oral cavity to remove debris and water. Because it is useful to avoid having the end sucking against the tongue, cheek or any other part of the oral cavity and so preventing removal of debris, the angled suction tip 24a enables the end of the tip to be rested or placed in various positions in the mouth on the apex of the tip 24a, while still leaving a gap between the angled end of the suction tip and the mouth, tongue or the like through which the debris, water and other matter can pass through the open end of the tip and into the vacuum line. Light beamed along the suction tip may all be reflected sideways from a 45 degree polished end so that it may be better to use a smaller angle or arrange that the end becomes a site of non-directional light emission.

A disposable, transparent retractor (see FIGS. 1a, 2, 3 or 8) is provided. It is connectable (by means of aperture 29 or by internal tube 21B—FIG. 8) to attach to the end of the suction attachment 20. The term “ecartor” is an equivalent name for a “retractor”. Retractor use is often required by the health professional carrying out the dental procedure, so it can be mounted or dismounted and rotated and placed on the suction tip at a suitable angle. Broad-surfaced retractors are used to deflect the cheek, the tongue, and/or other portions of the mouth away from a treatment site, incidentally avoiding possible injury from sharp or rotating tools. A retractor may be made entirely of a water-clear material, but is preferably tinted yellow (minus-blue) or orange (minus-blue and minus-green) as a built-in filter by addition of dyes for use in assessment of induced fluorescence. The filter is selected to prevent the user from seeing any excitatory light but transmits resulting fluorescence, if any. One source of dyes of this type for use in moulded retractors is “Epolin” (Newark, N.J.) who supply dyed pellets suitable for blending into the feed plastics used to make an injection-moulded retractor. The pellets include a selected dye of a range called Luminate™)

Optionally the retractor is moulded with or includes a convex profile, serving as a magnifying lens 32, that can be used by the dentist or dental assistant to more easily view details of the treatment site or any part of the oral cavity that is being examined: Otherwise the aperture 32 is optically a simple plane window free of any lens-like effects. Both sorts could be supplied in each set of tip and retractor. Another version of retractor as shown in FIG. 8 has at one end a hollow tube 21B that will fit inside the suction tip, 24. The retractor 21 is at an angle such as 45 degrees to the hollow tube 21b or the aperture 29 (FIG. 2), for placement over a complementary end of the suction tip. The window is optionally tinted as previously described. Retractor 21b fits into the interior of the central tube 24a of the suction tip 25, whereas the more preferred retractor 21 fits over the outside of a suction tip by means of a hexagonal or octagonal tapered hole that conforms to the shape of the most distal portion of the suction tip.

A recent embodiment of the invention is shown in exploded view in FIG. 8, which shows only half the shell of the body 10 with a front end 12, a light emitting assembly 16a, and some options for suction tips at 20. Two retractors are shown, at 21 and 21A. The former type fits over a perhaps octagonal suction tip, while the latter fits inside the central duct using snugly fitting tube 21B. The array of LEDs shown at 16A are surface-mount types, while FIG. 8A shows at 16B an array of conventional leaded LEDs and a heat sink/holder 16. The coupling 11 leads to the transfer pipe 15, to be coupled in turn to an internal duct 20a of a suction tip. Transfer pipe 15 is shown bent because it is made of flexible and replaceable materials and because it fits between other components. Cooling flow past the LED light sources comprises air pulled by the vacuum line through one-way valves 54A and 54 (at or near coupling 11) fitted to tube 52, drawn from within the shell 55 that surrounds and shrouds the light sources. One-way valve or valves 54, 54A are preferred in order to prevent saliva or the like from entering the electronics or the machine as back-flow through tube 52. This cooling flow may be in use throughout operation or may be activated only if components are overheated. A window 58 retains air flow and may prevent inadvertent entry of fluids. We have found quartz/silica to be suitable: it is transparent, strong and long-lasting yet not expensive. (Red Optronics, Mountain View, Calif., USA). Planar or conical (for light deflection) types of window may be used.

In this prototype, white and selected blue, deep blue and UV LEDs are used. If the measurement option is to be used, white light is preferably made or simulated by non-fluorescent means such as by use of red, green and blue LEDs run either simultaneously or turned on in a rapid sequence. This method of making white light also has the advantage that for example the green component can be enhanced for improved contrast where blood is involved. In the measurement option, blue/UV lamps are sometimes switched on and off rapidly under control of block 43. A circuit board 13a supports components used in control of emitted light and optionally for measurement of reflected light, and holds a microprocessor circuit and display means 13d, placed behind a sealed window, to display results to a user. The USB connector 13U is accessible and may be used to retrieve measured data, to download improved programmes, and/or to recharge the batteries 13b.

FIG. 7 is a block diagram of the electronics included in the dental tool. We first assume the simple mode of use, illumination but without built-in measurement; comprising only the parts on the left of the dotted line 46. (Tools may be made with various capabilities). Block 13c is a user control button or other ergonomically usable means which causes the mode of operation to switch between (a) off, (b) white light, and (c) blue/UV light. 13B represents a source of power such as rechargeable batteries. Since the forward voltage of some LED lamps is over 2.5 volts, a DC-DC voltage-raising power supply is used, such as one raising an input of 1.2/2.4 V to an output of 4 or 5 volts by boost conversion or another step-up method. Block 41 represents lamp current control and perhaps holds that boost converter, and block 16 represents an array of light emitting diodes.

The parts located to the right of the dotted line 46 are also used during the more complex mode of use, in which built-in measurement is operational. Block 43 represents computation means (optionally including data memory facilities) and display driver means for display 44. Typically block 43 would be a BASIC “Stamp” or similar microprocessor; the exact type is immaterial as long as it is compact, programmable, capable of the required tasks (including USB interface, display driver, and light control, and includes analogue to digital conversion means for reading photodiode output. (We assume the usual microprocessor support devices: resonators, power boost transistors, external memory and so forth). Block 42 comprises one or more photodiodes plus amplifying means (and perhaps microprocessor-controlled photocurrent integration means) for the detection of returned fluoresced light. A preferred photodiode is sensitive to as short as orange to green light (but not to the fluorescence-inducing light, which sensitivity may be controlled in part with the help of filters). Built-in measurement involves receiving fluorescence-emitted light from the adjacent intra-oral surface back through the end of the suction tip to as far as the electronic circuit board, and measuring the amount of light. Light pipes are inherently bidirectional. As long as the equipment itself does not include material capable of exhibiting fluorescence then some of the light picked up by the tip is fluorescing light from the tissue.

To describe the principle of operation, the blue/UV lamp or lamps are energised in about a 50% duty cycle at a rate of perhaps about 90 Hz or 35 Hz, so that mains supply-related flicker or mains-induced interference is not likely to interfere badly with the measurements. The light resulting from tissue autofluorescenee is returned down the suction tip and into the at least one photodiode. During the alternating periods when the blue/UV lamps are not on, light perceived by the photodiode must be extraneous and may be subtracted (using the microprocessor, or analogue electronics) from the light perceived when the blue/UV lamps are on. The entire hand tool is preferably used inside a clean or sterile shield since (at least at present) it is unlikely to be suitable for autoclaving or boiling or chemical sterilisation.

The user may select between an illumination mode suitable for general intra-oral examination and a fluorescence-based screening mode for oral disease. Any of the versions of disposable attachments may be used. In pure inspection modes of operation, the solid version of suction tip may be used. In general dental practice and for the purpose of suctioning and ejecting saliva, blood and debris, the user would take a fresh hollow square or round hollow attachment and insert it into the first light guide, and then connect the hand tool to a vacuum source. The switch is then turned to an illumination mode. In both situations the practitioner would use a fresh clear retractor to manipulate tissue, lift or push away the tongue or the cheek away from the teeth. Where an oral cancer detection or screening mode is of particular importance, the user may insert a solid examination “suction” tip that serves as a light guide, along with a retractor having a built-in filter, and insert it into the front of the hand tool. Either simple inspection under white or fluorescence-activating light, or a measurement procedure is then begun. Even if a ducted suction tip is not use, the connection to a vacuum source is still useful because the invention uses the resulting flow of air to cool the light source and the hand tool.

The different optical properties, particularly but not solely manifested as variations in colour or intensity of autofluorescence, may be seen as a difference in light reflectance between healthy and diseased tissue through the intra-oral window plus filter that is provided as part of the retractor, which assists with recognition of abnormal changes or diseased tissue.

As screening begins, the user may exert pressure on a suspected lesion, using the absorption filter/retractor. Under the influence of pressure, any healthy surrounding tissues will blanch more easily and any carcinomas and associated lesions will tend to remain unchanged. The differences in autofluorescence are often relatively clear and can be observed through the magnifier of the intra oral absorption filter/retractor. Further action should at least include making a permanent record of any suspected abnormality, and would involve the appropriate steps for preservation of patient health.

Variations

Infra-red mode: Noting that the combination dental hand tool provides a capacity to generate and to detect light over a wide range, including infra-red light, a variation of the basic tool comprises (a) one or light-emitting diodes that generate red and/or infra-red light by use of suitable light-emitting diodes, a (or the same) photodiode or phototransistor or other light sensor (which are usually inherently capable of sensing near infra-red light), and means to assess the relative proportion of visible to infra-red light that is returned from tissues adjacent to the distal end of the suction tip. Trials to date have indicated that an altered amount of reflectance of infra-red light is a correlate of abnormal tissues, such as hyperplasia, neoplasia and the like. Accordingly, a combination dental hand tool with infra-red reflectance capability may include an extra mode accessed by means of control switch 13c pressed one more time, to enter an infra-red or an infra-red versus visible measuring mode of operation, or it may be found more convenient to combine the use of infra-red light as well as visible returned light when assessing tissue fluorescence in order to minimise the complexity of use. Infra-red light may have a selective quenching effect on some forms of tissue autofluorescence, although investigations are not yet complete.

This aspect could be extended to include relatively narrow-band reflectance measurement in any or all of several infra-red bands, red, orange, yellow and green light, as well as measurement of induced fluorescence, simply by including light-emitting diodes of the selected colours. Each colour would be driven and measured in sequence. Since there is no colour difference between the emitted and received light, low scattered light within the optical paths is required for this aspect.

Clearly, a variant of the main body perhaps having simplified electronics,with a suction tip and retractor could be provided as a unit including one-time-use batteries such as for military field use—just making white light. The internal illumination function is a significant advantage for field use.

The suction tip may include in-situ LEDs or organic LEDs powered by embedded wires leading through a connector to the body of the tool, if such devices become suitable.

A mode of use involving measurement may include periodic interruption of white light during which time the blue on/off cycle is carried out several times before persistence of vision effects make the user aware that the white light is flickering. As a result, fluorescence measurement may occur during the normal use of white light.

The dentist or hygienist could be provided with an eye shield or eyeglasses including an appropriate long-pass filter for use during fluorescence-based examination, so that a retractor may not need to be used, or so that a retractor that otherwise requires to be tinted, does not need to be tinted.

A camera may be used to to acquire an image of the fluorescence emitted from the tissue under blue light, as well as an image of green to red tissue reflectance. The image may then be displayed and/or enhanced on a video monitor or computer in real time and may be stored for comparison with the lesion at a later date.

The main body could derive its power from a connection to external wiring that is also used to carry data to a nearby computer.

A “Bluetooth”® or similar local wireless link could be used to pass fluorescence information in real time to a nearby computer.

### ADVANTAGES AND COMMERCIAL APPLICABILITY

A single tool having multiple functions is significantly preferable over a range of tools each performing a single function. Reasons include:

1. Less space is occupied inside the patient's mouth and through the aperture formed by the patient's lips because the total number of tools in use at one time is reduced.

2. Quicker and easier to perform operations since tools do not have to be withdrawn, placed on a tray, whereupon another tool can be picked up, directed to the treatment site, and finally placed in the right orientation (by which time a bleeder or saliva or the like may have obscured the site so that it has to be re-exposed). This aspect saves time, difficulties such as limited duration of anaesthetic, patient stress, and allows more patients to be seen in a day.

3. The present invention is easily incorporated into an existing dental surgery without any changes to the existing equipment, since it plugs on to the free end of the existing flexible suction line and can be recharged through (for example) a USB connection to a computer, or a USB-style connector to a power pack.

4. Use of a solid suction tip as a disposable item avoids problems related to introducing a fibre optics bundle into the mouth; too costly to make disposable and too likely to trap organisms to be re-used safely.

5. The present invention provides means to screen a patient's oral cavity for abnormal tissues during a standard procedure, thereby allowing early cancers to be seen more readily (thanks to the use of tissue or added fluorescence).

6. The present invention may be used by a dental hygienist (who also use suction tips) who would be able to alert a corresponding dentist in case any clear or suspicious sign of abnormality is seen.

7. The present invention may be used in situations where a conventional surgery having overhead lighting is absent. (The suction function could be produced by a foot pump in a refugee camp or the like).

Finally, it will be understood that the scope of this invention as described and/or illustrated herein is not limited to the specified embodiments. Those of skill will appreciate that various modifications, additions, known equivalents, and substitutions are possible without departing from the scope and spirit of the invention as set forth in the following claims.

1-13. (canceled)

14. A combination dental hand-held tool for carrying out several separate or concurrent purposes by a user during a dental examination and/or treatment procedure carried out within the oral cavity of a dental patient, characterised in that the tool includes (a) a first reversible connection means for connecting to a first end of an elongated light-carrying rod made of a transparent, refractile water-clear plastics material; the connection means serving, when in use, to couple light into the first end of the rod to be carried along the rod and emitted inside the dental patient's oral cavity or mouth at or near a second end of the rod, and to receive light including light carried back along the rod from the dental patient's oral cavity to the first end; (b) a second reversible connection means capable of coupling an existing vacuum line through a passage within the body of the tool up to the first reversible connection means, thus providing the tool with a capability for a suction function; (c) means capable of illuminating the light-carrying rod comprising at least two separately operable light sources contained within the tool; one source emitting, when in use, white light and each of the one or more further sources emitting, when in use, a selected band of wavelengths selected from the range between and including infra-red light and ultra-violet light, some of which bands are capable of inducing tissue fluorescence; and (d) means capable of detecting and measuring the light carried back along the rod from the dental patient's mouth, capable when in use of reporting that light that has been emitted by tissue fluorescence or reflected from the dental patient's oral cavity as a result of illumination with light having a defined range of wavelengths.

15. A combination dental hand-held tool as claimed in claim. 14, characterised in that each tool is provided with more than one type of elongated light-carrying rod, the types including a solid rod adapted for use in inspecting the interior of the patient's mouth with light having a defined range of wavelengths, and a tubular rod, open at both ends and capable when in use of providing a combination of illumination and suction within the interior of the patient's oral cavity.

16. More than one type of elongated light-carrying rod as claimed in claim 15 for use with a combination dental hand-held tool, characterised in that the or each type of rod is provided in a pack holding one or more disposable rods ready for use.

17. A retractor capable of use with any one type of elongated light-carrying rod as claimed in claim 15, characterised in that the or each retractor is reversibly attachable to the second end of the rod and comprises a rigid, outwardly projecting flap held to one side of the second end of the light-carrying rod and capable of pushing and holding soft parts of the patient's oral cavity away from a treatment area; the or each retractor also including a transparent window so that when in use the user may view that part of the interior of the patient's oral cavity that is situated directly behind the retractor and is provided in a pack containing one or more disposable retractors ready for use.

18. A retractor as claimed in claim 17 characterised in that the window of the retractor is coloured so that the window selectively blocks that light used to induce tissue fluorescence while transmitting the light emitted by fluorescence, so that the user may directly inspect the dental patient's oral cavity for visible fluorescence or a change of visible fluorescence and is provided in a pack containing one or more disposable retractors ready for use.

19. A retractor as claimed in claim 17 characterised in that the window of said transparent retractor includes magnifying means.

20. A tool as claimed in claim 14, characterised in that the measuring means provided within the tool for measuring, when in use, light received from the light-carrying rod includes means whereby the light source capable of inducing fluorescence is turned on then off in a cyclic manner and the measuring means is controlled so as to subtract each first reading of received light taken when the source is off from a second reading obtained when the source is on, hence rendering measurements of fluorescence relatively independent of interference.

21. A tool as claimed in claim 14, characterised in that the measuring means comprises digital computing means within the hand tool, and the reporting means comprises a user-readable output upon a display means incorporated within the combination hand-held tool.

22. A tool as claimed in claim 21, characterised in that the measuring means is capable from time to time of transmitting readings to an external computer through a compatible wired or wireless connection.

23. A retractor as claimed in claim 18 characterised in that the window of said transparent retractor includes magnifying means.
